(19) United States
(12) Patent Application Publication
(10) Pub. No.: US 2018/0177426 A1
(43) Pub. Date: Jun. 28, 2018

(54) Device Based on Virtual Reality Interactive Technology and Real-time Monitoring Technology of Brain Function

(71) Applicant: South China University of Technology, Tianhe Guangzhou (CN)

(72) Inventor: Xiangmin XU, Tianhe Guangzhou (CN)

(73) Assignee: South China University of Technology, Tianhe Guangzhou (CN)

(21) Appl. No.: 15/735,067

(22) PCT Filed: Dec. 21, 2016
(86) PCT No.: PCT/CN2016/111388

§ 371 Date: Dec. 8, 2017

(30) Foreign Application Priority Data
May 10, 2016 (CN) 201610304124.8

(51) Int. Cl. A61B 5/0484; A61B 5/00; A61B 5/16; A61B 5/11; A61B 5/026; A61B 5/0205; A61B 5/0488; G16H 40/63

(52) U.S. Cl. CPC A61B 5/04842; A61B 5/7445; A61B 5/165; A61B 5/7246; A61B 5/7275; A61B 5/0245; A61B 5/163; A61B 5/026; A61B 5/0205; A61B 5/0488; G16H 40/63; A61B 5/1126

(57) ABSTRACT

The present invention provides a device based on virtual reality interactive technology and real-time monitoring technology of brain function, which relates to the field of medical treatment and intelligent device. It comprises a virtual reality interactive module, a human body data collecting module, a data synchronization module, and a control module for data processing and analysis; wherein the virtual reality interactive module is responsible for providing specific virtual reality contents to induce specific emotion of a user and for collecting an interactive behavior data of the user when interacting with the virtual reality interactive module; the human body data collecting module is responsible for collecting a real-time physiological sign data of the user; the data synchronization module is responsible for synchronizing the real-time collected interactive behavior data and the real-time collected physiological sign data of the user; the control module for data processing and analysis is responsible for analysing and processing the original physiological signs data and finally obtains, in connection with big data, a health state of the brain function of the user. The present invention is convenient and flexible for operation, convenient for scientific research, and can also be used for self-assessment, which helps to save medical resources and improve the level of diagnosis and treatment.

### TECHNICAL FIELD

The present invention relates to the field of medical treatment and intelligent device, and specifically to a device based on virtual reality interactive technology and real-time monitoring technology of brain function.

### BACKGROUND

Virtual reality interactive technology has been developed rapidly in recent years. It brings immersive experience to users, and it has, in addition to visual perception, auditory perception, tactile perception, motion perception, and it even includes taste perception, smell perception, and so on. The ideal virtual reality should have all perceptual functions that human beings have. Virtual reality technology is not only used in the aspect of entertainment, but also has a relatively high value in the aspect of medical treatment. There have been many teams using virtual reality technology to make a try in the aspect of medical treatment.

Human has never stopped researching on brain science, but there are some difficulties in the research due to the complexity of the human brain. And with the rapid development of society, people are under increasing pressure, and more and more people are disturbed by a variety of mental and psychological problems. But they lack the means to recognize their own more internal health state, which includes health state of the brain function, and their symptoms have been more serious by the time they are discovered.

Virtual reality interaction technology provides new means and methods for scientific research on human brain. The present invention applies virtual reality interaction technology to the aspects of brain function monitoring and researching. A user can monitor the health state of the brain function of a person by using the present invention; the present invention can provide more objective data and auxiliary information in the process of the diagnosis and treatment of mental illness; and the present invention also provides a new research means in the aspect of scientific research on brain.

### SUMMARY OF THE INVENTION

The objective of the present invention is to provide a device based on virtual reality interactive technology and real-time monitoring technology of brain function. The device provides a user with specific virtual reality contents to induce emotion changes of the user by virtual reality interactive technology; collects an interactive behavior data, and a physiological sign data, such as brain wave, cerebral blood flow and so on; processes and analyzes these data; and obtains feedback from the user by analysis. The device can provide the user with data related to the brain function and assessment results of health state of brain function, which facilitates long-term recording and observation of the user's brain function and achieves brain function monitoring.

The technical solution adopted by the present invention is as follows.

A device based on virtual reality interactive technology and real-time monitoring technology of brain function, comprising a virtual reality interactive module, a human body data collecting module, a data synchronization module, and a control module for data processing and analysis, wherein

the virtual reality interactive module provides virtual reality contents to induce emotion changes of a user and simultaneously collects an interactive behavior data of the user and sends the collected data to the data synchronization module;

the human body data collecting module collects a real-time physiological sign data of the user and sends the collected data to the data synchronization module;

the data synchronization module synchronizes the real-time interactive behavior data and the real-time physiological sign data of the user and sends the synchronized data to the control module for data processing and analysis; and

the control module for data processing and analysis processes the original physiological signs data; performs a comparative analysis between the physiological sign data and the normal data, and between the interactive behavior data with and the normal data; obtains a state of the user's brain response to current virtual reality contents based on the degree of similarity from the comparative analysis; controls the virtual reality interactive module to present new virtual reality contents to induce the emotion changes of the user to enter into next round of detection; and circulates successively until all the provided virtual reality contents are tested; the normal data refers to the physiological sign data and the interactive behavior data of normal people which are produced when they are influenced by the same virtual reality contents.

Furthermore, the interactive behavior data comprises eye movements, head movements, voice or hand movements.

Furthermore, the physiological sign data collected by the human body data collecting module comprises the physiological sign data of brain wave, cerebral blood flow, pulse, electrocardio or myoelectricity.

Furthermore, the processing of the control module for data processing and analysis to the original physiological signs data comprises sampling, quantization, coding, smoothing or format unification.

The virtual reality interactive module can provide an immersive experience to the user. The virtual reality interactive module has functions including, but not limited to, virtual reality contents display and interactive behavior monitoring. The virtual reality interactive module can provide the user with specific virtual reality contents and induce specific emotion changes of the user. In the process of interaction, interactive behavior data of the user will be collected, which includes but is not limited to, the eye movements, head movements, voice or hand movements.

The human body data collecting module collects, in real time, the user's physiological sign data when interacting with the virtual reality interactive module; and the physiological sign data includes but is not limited to, brain wave, cerebral blood flow, pulse, electrocardio or myoelectricity.

The data synchronization module provides data synchronization service for the real-time collected physiological sign data and the real-time collected interactive behavior data, and sends the data to the control module for data processing and analysis.

The control module for data processing and analysis has functions including, but not limited to, data processing, data analysis, and virtual reality interactive module controlling. This module performs a data processing to the received synchronized physiological sign data and interactive behavior data, wherein the processing contents includes, but is not limited to, sampling, quantization, coding, smoothing and format unification. This module performs a comparative analysis between the collected physiological sign data and the anticipated normal data (i.e., the physiological sign data of normal people when influenced by the same virtual reality contents); simultaneously analyzes interactive behavior data; and analyzes the state of the brain function by combining both of them. When the user uses the device, this module controls the virtual reality interactive contents based on the changing state of the user's physiological signs data and interactive data.

The device based on virtual reality interactive technology and real-time monitoring technology of brain function can communicate with the local server or remote server to achieve functions such as data storage, data analysis, results display, remote control and so on. Thus a doctor can remotely provide medical services for a user.

Compared with the prior arts, the present invention includes at least the following advantages:

The present invention provides an easy to use device for monitoring brain function, which is suitable for being used in home and hospital. It brings immersive experience to users by virtual reality interactive technology, enables the user to accept the artificial mental influence and evokes specific emotion of the user. These controllable influence and predictable emotional fluctuation provide new methods for monitoring brain function. The device is simple to use, and can be used by an ordinary user to perform health monitoring of his or her own brain function, which helps the user to monitor the health state of the brain function of a person. In the aspect of medical diagnosis and treatment, the device benefits diagnosis and recovery of mental and physical problems, which can improve the accuracy of diagnosis. In the meantime, it saves medical resources, improves the level of diagnosis and treatment, and helps to form a diagnosis and treatment standard and guideline, such that diagnosis and treatment of mental illness becomes more standardized and more formal. In the aspect of the researching on brain science, the device provides new researching methods, which helps to find the relevant markers and relevant mechanisms, thereby improving the effect of health improvement of the observed.

### BRIEF DESCRIPTION OF THE DRAWINGS

FIG. 1 shows a device based on virtual reality interactive technology and real-time monitoring technology of brain function of the embodiment.

### DETAILED DESCRIPTION OF THE PREFERRED EMBODIMENT

The present invention is further described hereinafter in connection with the accompanying drawings

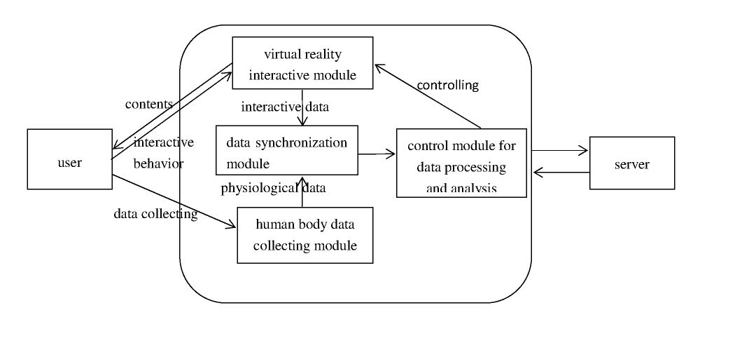

As shown in FIG. 1, a device based on virtual reality interactive technology and real-time monitoring technology of brain function, mainly comprises four modules: a virtual reality interactive module, a human body data collecting module, a data synchronization module, and a control module for data processing and analysis.

The virtual reality interactive module has at least the functions of virtual reality contents display and interactive behavior monitoring. A user needs to wear the device under circumstances of ensuring the security. After the wearing is completed, the virtual reality interactive module can provide specific virtual reality contents under the control of a user or a doctor, and induce the specific emotion changes of the user by immersive virtual reality contents. As the device supports to connect with a remote server, the controller of the device can be a doctor performing remote control. In the process of interaction, the virtual reality interactive module will monitor all the interactive behavior of the user, and will collect the interactive behavior data; wherein the interactive behavior data includes but is not limited to, eye movements, head movements, voice or hand movements.

In the process that the user interacts with the virtual reality interactive module, the human body data collecting module also collects the user's physiological sign data, which includes but is not limited to brain wave, cerebral blood flow, pulse, electrocardio or myoelectricity, to analyze the user's emotional fluctuation.

As both the physiological sign data and the interactive behavior data are collected in real time, and interactive behavior have a certain connection with the change of the physiological sign data, the synchronization of these two data is the key link of the device. The data synchronization module simultaneously receives the collected physiological sign data and the collected interactive behavior data, and synchronizes these two data to facilitate the subsequent processing and analysis.

The synchronized physiological sign data and the synchronized interactive behavior data are given to the control module for data processing and analysis. The control module for data processing and analysis has functions including, but not limited to, data processing, data analysis, and virtual reality interactive module controlling. This module performs a data processing to the physiological sign data and the interactive behavior data, wherein the processing contents includes but is not limited to, sampling, quantization, coding, smoothing and format unification. After the data are processed, this module performs a comparative analysis between the collected physiological sign data and the anticipated normal data (i.e., the physiological sign data of normal people when influenced by the same virtual reality contents); analyzes the differences in the data; simultaneously analyzes the interactive behavior data; and analyzes the state of the brain function by combining both of them. When the user uses the device, this module controls the virtual reality interactive contents based on the analysis results of the user's physiological signs data and interactive data.

The device can be connected to a server (which could be a local server or a remote server). The data and analysis results can be stored in the server, and the server can also control the operation of the device. The device has the ability to connect with a remote server, which also makes remote medical treatment possible. A doctor can remotely observe a user's status, to provide medical services for the user.

What is claimed:

1. A device based on virtual reality interactive technology and real-time monitoring technology of brain function, characterized in that, the device comprises a virtual reality interactive module, a human body data collecting module, a data synchronization module, and a control module for data processing and analysis, wherein
the virtual reality interactive module provides virtual reality contents to induce emotion changes of a user and simultaneously collects an interactive behavior data of the user and sends the collected data to the data synchronization module;
the human body data collecting module collects a real-time physiological sign data of the user and sends the collected data to the data synchronization module;
the data synchronization module synchronizes the real-time interactive behavior data and the real-time physiological sign data of the user and sends the synchronized data to the control module for data processing and analysis; and
the control module for data processing and analysis processes the original physiological signs data; performs a comparative analysis between the physiological sign data and the normal data, and between the interactive behavior data with and the normal data; obtains a state of the user's brain response to current virtual reality contents based on the degree of similarity from the comparative analysis; controls the virtual reality interactive module to present new virtual reality contents to induce the emotion changes of the user to enter into next round of detection; and circulates successively until all the provided virtual reality contents are tested;
the normal data refers to the physiological sign data and the interactive behavior data of normal people which are produced when they are influenced by the same virtual reality contents.

2. The device based on virtual reality interactive technology and real-time monitoring technology of brain function according to claim 1, wherein the interactive behavior data comprises eye movements, head movements, voice or hand movements.

3. The device based on virtual reality interactive technology and real-time monitoring technology of brain function according to claim 1, wherein the physiological sign data collected by the human body data collecting module comprises the physiological sign data of brain wave, cerebral blood flow, pulse, electrocardio or myoelectricity.

4. The device based on virtual reality interactive technology and real-time monitoring technology of brain function according to claim 1, wherein the processing of the control module for data processing and analysis to the original physiological signs data comprises sampling, quantization, coding, smoothing or format unification.
